SEQUENCE LISTING (2 sequences)

SEQ ID NO: 1 - DNA, 17 bp, Artificial Sequence
  Synthetic DNA sequence used to form a monolayer on a gold surface
tatgcagaaaatcttag

SEQ ID NO: 2 - DNA, 26 bp, Artificial Sequence
  Synthetic DNA sequence used to form a monolayer on gold nanoparticles
ctaagattttctgcatagcattaatg

(19) United States
(12) Patent Application Publication
(10) Pub. No.: US 2006/0194228 A1
(43) Pub. Date: Aug. 31, 2006

(54) DNA sequence recognition

(75) Inventors: Andrei Rakitin, Etobicoke (CA); Andrey Ginovker, Toronto (CA); Alexander E. Dudelzak, Ottawa (CA)

Correspondence Address: MARKS & CLERK, P.O. BOX 957, STATION B, OTTAWA, ON, K1P 5S7, CA

(73) Assignee: Grade BioSense Inc., Ottawa (CA)

(21) Appl. No.: 11/334,540

(22) Filed: Jan. 19, 2006

Related U.S. Application Data
(60) Provisional application No. 60/644,815, filed on Jan. 19, 2005.

(51) Int. Cl. C12Q 1/68; C12M 1/34; G01F 1/64

(52) U.S. Cl. 435/6; 977/924; 435/287.2; 205/777

(57) ABSTRACT

To detect the presence of a match of a target DNA base sequence with a probe DNA base sequence, a single strand of the probe DNA base sequence is prepared. One end of the single strand probe DNA base sequence is linked to an electrode and the other end to a nano entity capable of exchanging charge with the DNA base sequence. The single strand of the target DNA base sequence is brought into contact with the single strand of the probe DNA base sequence, and the change in the physical properties of the probe DNA base sequence upon hybridization detected.

### CROSS REFERENCE TO RELATED APPLICATION

This application claims the benefit under 35 USC 119(e) of prior U.S. Provisional application No. 60/644,815, filed Jan. 19, 2005, the contents of which are herein incorporated by reference.

### FIELD OF THE INVENTION

This invention relates to a method and apparatus recognizing a specific probe DNA sequence by comparing it with a target DNA base sequence.

### BACKGROUND OF THE INVENTION

The detection of DNA hybridization is becoming of increasing importance in the diagnosis and treatment of genetic diseases, the detection of infectious agents, and forensic analysis. As is well known DNA consists of a double helix of complementary base pairs. Hybridization refers to the process where complementary matching single strands of DNA are brought together. In forensic analysis, for example, the object is to determine whether two DNA samples match. This can be achieved by splitting each sample into single strands and bringing the resulting single strands together. In the event of a match, the single strands will hybridize, or combine, to form a double-stranded molecule.

A number of techniques exist in the prior art for detecting hybridization and are described, for example, in Electrochemical DNA Sensors, T. G. Drummond, M. G. Hill, and J. K. Barton, Nature Biotechnology, Vol. 21, No. 10, Oct. 2003; and Electroactive Beads for Ultrasensitive DNA detection, J. Wang, R. Polsky, A. Merkoci, and K. L. Turner, Langmuir 2003, 19, 989-991. These techniques rely on the fact that the binding event creates a signal that can be detected in some way. One way is to use an optical readout, where changes in optical properties are observed. Another way is to use an electrochemical readout, where the changes in the electrical properties of a solution containing reporter molecules are detected. In one such technique, guanine residues in DNA reduce a metal complex. The detected signal represents the amount of guanine available for oxidation.

In another technique, photochemical detection is used. In this technique, immobilized probe DNA is hybridized to target DNA, which is subsequently hybridized to a reported strand labelled with CDS nanoparticles. Exposure of the Cds-DNA aggregate to visible blue light triggers a photoelectrochemical current between the CdS nanoparticle aggregate and a gold electrode.

These prior art techniques deal however with bulk solutions, and they are generally insensitive to slight mismatches in the DNA strands, that is when only partial hybridization occurs.

### SUMMARY OF THE INVENTION

The invention provides a method for detecting hybridization of a specific sequence of base pairs in a single strand of DNA, which is sensitive to partial matches based on a change of physical properties of a DNA molecule in the presence of a hybridization event.

According to the present invention there is provided a method of detecting the presence of a match of a target DNA base sequence with a probe DNA base sequence, comprising preparing a single strand of the probe DNA base sequence; linking a first end of said single strand probe DNA base sequence to an electrode and a second end to a nano entity capable of exchanging charge with the DNA base sequence; bringing a single strand of the target DNA base sequence into contact with the single strand of the probe DNA base sequence; and detecting a change in a physical property of said probe DNA base sequence due to hybridization in the event of a match of and said target DNA base sequence.

It is of course possible to arrange more than one single strand of DNA in a chain with nano entities between them.

In one embodiment the charge transfer is initiated by optically exciting the entity. The current can be detected when a voltage is applied to the electrode. In another embodiment, an electric voltage is applied to create a nanoshuttle as will be explained in more detail below.

Although reference is made to optical excitation, it will be understood that the invention is not limited to visible light. An electromagnetic radiation of suitable wavelength to excite the excitable entity and cause it to transfer charge can be employed.

One embodiment of the invention is based on the fact that single-stranded, i.e. non-hybridized DNA molecules exhibit a band gap in the density of electronic states of a few electron-volt width, with the electric current less than a few pA flowing through the DNA molecule. Thus, an event of hybridization, i.e., the formation of a double-stranded DNA molecule can be accompanied by an increase in the electric current flowing through the molecule kept under otherwise constant electrical, chemical, etc. external conditions, provided that the measurement and the sample preparation do not deform the original DNA structure. In another embodiment the change in elasticity of the DNS strand is detected by detecting the resonant frequency of oscillation.

The electrical measurements should be conducted on non-deformed DNA molecules. Deformation of a double-stranded DNA may result in a change of DNA electronic structure, with a band gap widening or opening in the density of DNA electronic states, leading to a drop in DNA conductivity and to a decrease in electrical current flowing through the DNA molecule in a range from nA to pA [A. Rakitin, International Symposium on DNA-based molecular electronics, IPHT Jena (Germany), May 13-15, 2004 (all the Symposium presentations are published on-line at http://faculty.une.edu/cas/jvesenka/researchpubs/jenanotesppt/dnasymposium.html)].

The nano entity may be, for example, a Fullerene molecule, a nanoparticle, particularly a semiconductor nanoparticle, or a redox-active molecule. A Fullerene molecule is a molecule consisting of carbon atoms.

When hybridization occurs, the strand of DNA becomes more conductive and allows charge to be transferred through the hybridized DNA molecule between the excitable entity and the electrode at the other end. In accordance with the principles of the invention, this current is detected and used to detect hybridization. Typically, the current through a single strand is in the order of pico amps, whereas the current through a hybridized double strand is in the order of nano amps.

Unlike the prior art, embodiments of the invention can detect the hybridization of a single strand of DNA. Moreover, the optically excited current flowing through the DNA molecule from the excitable entity depends on the degree of matching between the two strands, and thus gives a measure of the extent to which the two strands match. Quantitative changes in the amplitude and modulation frequency of the current flowing through the DNA molecule report on the DNA hybridization success or failure and on the number of base pair mismatches in the case of the DNA partial hybridization.

In one embodiment, the single strand of probe DNA is in a solution containing divalent metal ions. As the two strands come together, metal ions form a metal-like chain inside the double-stranded DNA so as to form a highly conductive path along the length of the DNA sequence.

In another embodiment, a pair of the single strand probe DNA base sequences is connected together through the nano entity, and the outer ends of the base sequences are connected to electrodes to form a nanoshuttle. In this embodiment, the change in resonant frequency of the nanoshuttle upon hybridization is detected from the current flowing through it.

In another aspect the invention provides a DNA detection apparatus, comprising a sensor with a surface forming an electrode; a single strand probe DNA base sequence having one end bonded to an electrode and another end linked to an entity selected from the group consisting of a fullerene molecule, a nanoparticle, and a redox active molecule; and a detector for detecting current flowing through the DNA base sequence upon hybridization.

### BRIEF DESCRIPTION OF THE DRAWINGS

The invention will now be described in more detail, by way of example only, with reference to the accompanying drawings, in which:

FIG. 1 is a schematic illustration of a first embodiment showing a single strand probe DNA sequence inserted between an excitable entity and an electrode; and

FIG. 2 is a schematic illustration of a second embodiment showing a pair of single stranded probe DNA sequences.

### DETAILED DESCRIPTION OF THE PREFERRED EMBODIMENTS

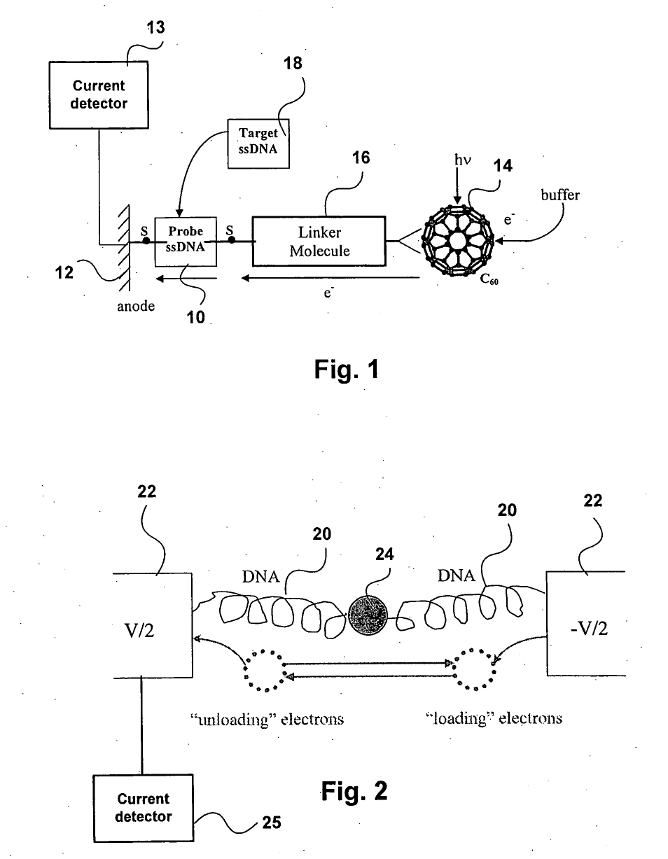

One of the possible measurement schemes is shown in FIG. 1. Here is single strand of probe DNA base sequence 10 is bonded to a gold electrode 12, forming an anode, and to a Fullerene molecule 14 by a linker molecule 16. A single strand of the target sequence 18 is brought into contact with the probe sequence. The Fullerene molecule is optically excited, and when a match occurs, the resulting current through the hybridized molecule can be detected. The sulphur atoms S are used to bind the ends of the DNA sequence to the electrode 12 and linker molecule 16. The current flowing upon the event of hybridization is detected by current detector 13. The fullerene molecule 14 can be optically excited by argon laser 15.

A photo-induced electron transfer at a gold electrode modified with a self-assembled monolayer of Fullerene (C₆₀) and a setup for electric current measurements used in these scheme were disclosed in a paper by Imahori et al. (Chem. Commun., 557-558 (1999)). The same paper teaches how to synthesize a polyalkanethiol linker 16 between the fullerene molecule and an S atom known to bind to a gold electrode. The technique of attaching DNA molecule to an S atom and to a gold electrode through sulphur-gold interaction was disclosed in a paper by Braun et al. (Nature, 391, 775-778 (1998)).

Semiconductor nanoparticles including but not limited to compounds such as CdS, PbS, ZnS can also be used instead of Fullerene molecules to allow optically excited electron transfer.

The single-stranded DNA sequence 10 interrupts the current flow between the fullerene molecule (or other optically excited nanoparticle) and electrode.

If the probe ssDNA molecule is short cut, a stable anodic photocurrent flows immediately after the fullerene hold in the buffer solution containing AsA as an electron sacrificer is irradiated at a wavelength about 350 nm with an argon laser.

An increment of the anodic photocurrent with an increase of positive bias to the gold electrode demonstrates the direction of current from the cathode (platinum counter electrode) to the anode through the buffer electrolyte. Placement of the probe ssDNA molecule between the fullerene and the anode leads to an almost complete suppression of photocurrent, which is due to the wide band gap in the density of ssDNA electronic states and the consequent low value of electrical conductivity/ high value of electrical resistance.

Adding a complementary single-stranded target DNAs into the buffer followed by hybridization with the probe DNAs results in a photocurrent increase due to a substantial narrowing of the electronic band gap and a consequent increase in the DNA conductivity upon conversion thereof from a single-stranded into a double-stranded form. It is of importance that the DNA molecules are kept in a solution rather than lay on a solid substrate because an interaction with the substrate may result in a DNA deformation, affecting in its turn the DNA electronic properties as described herein above.

### EXAMPLE 1

A polished n-type single crystal (111) silicon wafers (MOTOROLA, resistance 10-15 Ohm cm⁻²) were cleaned by boiling in ethanol (Merck, Pro-Analysis) for 20 min and dried in nitrogen. The wafers were immediately placed in an electron beam evaporator (Edwards, Auto 306 Turbo) equipped with a thickness monitor (Edwards FTM7). The deposition was carried out at a base pressure of 5×10⁻⁶ mbar. A 1 nm chromium adhesive layer was deposited at a rate of 0.01 nm/s. The rate of the gold deposition (100 nm thick) was 0.01-0.03 nm/s. Prior to the DNA adsorption the gold substrates were first rinsed twice in boiling ethanol for 20 min. After drying in nitrogen they were immersed in hot piranha solution (3:1 H_{—}2SO_{—}4:H_{—}2O) for 10 min. They were then thoroughly rinsed with ultra-pure water (Millpore, 18 MOhm).

The 3′-thiolated DNA oligonucleotides were kept in their oxidized form-(CH_{—}2)_{—}3—S—S—(CH_{—}2)_{—}3—OH in order to protect the thiol group from undesired oxidation products or dimerization. Prior to adsorption, DNA was incubated with 10 mM of the reducing agent Tris(2-carboxyethyl)phosphine (TCEP) in 100 mM Tris-HCl, pH 7.5. The mixture was incubated at room temperature for several hours to allow complete reduction of the disulfide bond. The DNA samples were then passed through a Bio-Rad BioSpin-6 column pre-equilibrated with the buffer (0.4 M NaH_{—}2PO_{—}4, pH 7.4). The final ssDNA concentration was adjusted to 10 μM. From 10 to 15 μl of the 10 μM-reduced DNA solution was pipetted onto a clean gold surface. The gold samples were then placed in a sealed Petri dish at 100% humidity. After 2 hours of adsorption the samples were rinsed (a 20 min incubation in the adsorption buffer). Rinsing was done three times. Then, the samples were thoroughly rinsed with sterile ultra-pure water. Samples were kept in sterile ultra-pure water and were dried in nitrogen right before characterization. The DNA sequence used to form a monolayer on the flat gold surface was 5′-TAT-GCA-GAA-AAT-CTT-AG-3′.

Gold nanoparticles (Aldrich-nominal of 10±3 nm diameter) were rinsed with deionized water by two centrifugations at 8000 rcf. After the water surfactant was removed, 200 μl of 10 μM of reduced thiolated ssDNA diluted in sterile deionized water was added to the gold nanoparticles. The mixture was stirred overnight at room temperature. Following incubation, the nanoparticles were rinsed with water and the Tris (0.025 M)-NaCl (0.2 M pH 7.5) buffer by one centrifugation cycle at 10000 rcf. The nanoparticle-ssDNA complex in 0.2 M buffer was agitated for 4 h at room temperature, followed by rinsing twice in Tris (0.025 M)-NaCl (0.4 M). The 5′CTA-AGA-TTT-TCT-GCA-TAG-CAT-TAA-TG-3′ sequence was used to form a monolayer on gold nanoparticles. From 10 to 15 μl of the nanoparticle-ssDNA diluted in a Tris ç were dropped onto the ssDNA monolayer on gold. The sample was placed in a sealed Petri dish at the 100% humidity as above. After 12 h of incubation each sample was rinsed three times for 20 min, with the Tris (0.025 M)-NaCl (0.4 M) buffer. Prior to characterization, each sample was rinsed with sterile deionized water to remove excess salt.

Attaching fullerene molecules to gold nanoparticles was accomplished as described by Imahori et al., Chem. Commun., 557-558 (1999). Photo-electrochemical measurements were carried out in an argon-saturated 0.1 M Na_{—}2SO_{—}4 solution containing 50 mM of ascorbic acid (AsA). The DNA-fullerene functionalized gold electrode was as the working electrode in conjunction with a platinum counter electrode and Ag/AgCl reference electrode.

Optical input was modulated with a chopper (SR 540, Stanford Research Systems) at 10 kHz. The output signal went through a lock-in amplifier (SR 530, Stanford Research Systems) and was eventually detected with an electrochemical analyzer (CH 660B, CH Instruments). Illumination (wavelength 403±7 nm, 6.6 mW/cm² power density) of the working electrode surface under a 2V voltage applied biasing but in absence of the complementary DNA chain results in no detectable anodic photocurrent (above the 1 pA detection threshold).

After addition of 10-15 μl solution containing the complementary ssDNA molecules (5′-CAT-TAA-TG-3′, 10 fM concentration) and 10 min incubation time, appreciable 3 nA anodic current was detected.

In another embodiment the electrical measurements are performed on metallic DNA molecules. Metallic DNA (M-DNA) is a DNA derivative in which imino protons have been selectively replaced with divalent metal ions having a d-electron shell. [J. S. Lee, L. J. P. Latimer, and R. S. Reid, Biochem. Cell. Biol. 71, 162 (1993) demonstrated that such a replacement takes place in the case of Zn²⁺, Ni²⁺, Co²⁺]. M-DNA retains the double helical structure of common DNA's anti-parallel strands, but with one important difference. In addition to a linear chain of base-pairs, the DNA sugar-phosphate backbone now encapsulates a linear chain of metal ions. It has been experimentally shown that a DNA can be converted into an M-DNA regardless of sequence of base pairs [Aich et al., J. Mol. Biol. 294, 477 (1999)]. The process is reversible, i.e., an addition of EDTA (ethylenediaminetetraacetic acid) quickly restores the original DNA. The divalent metal ions can be added to the hybridized DNA, or alternatively added after hybridization. In either case, the metal ions become intercalated between the DNA base pairs and enhance conductivity.

Direct electrical measurements conducted on M-DNA molecules revealed their metal-like electrical behavior [Rakitin et al., Phys. Rev. Lett. 86, 3671 (2001)]. The same paper teaches that DNA molecules that have not undergone the DNA—M-DNA transformation exhibit a band gap in the density of electron states and, hence, a very low electrical conductivity even in presence of metal ions adsorbed on the DNA surface and dissolved in the DNA buffer.

A metal ion can substitute an imino proton and go inside the DNA double-helix, i.e., can occupy position between two base pairs, if and only if these base pairs match. Any base-pair mismatch, i.e., their incomplete hybridization, results in an interruption in the DNA encapsulated linear metallic chain, with metal ions being adsorbed on the DNA surface or being dissolved in the buffer.

One of possible detection schemes for this embodiment includes the scheme of FIG. 1 with the difference that now the DNA buffer contains the divalent metallic ions (Zn, Ni, Co) at a concentration and pH that would result in the M-DNA formation (conditions required for M-DNA formation are disclosed by Rakitin et al.) if the target DNA and probe DNA strands match-hybridized with each other. If no hybridization takes place, almost no photocurrent flowing to the anode can be observed because the target ssDNA exhibits poor conductivity and by itself can not be converted into the M-DNA form.

In the case of a complete hybridization, an observed high value of photocurrent clearly indicates the perfect match between the target and the probe DNA base pairs enabling continuous linear metallic chain formation as described above. In a case of partial hybridization, mismatches between the probe and the target DNA base-pairs result in the interruptions of metallic chain indicating the places of mismatch. These interruptions result in lower conductivity and, hence, lower photocurrent and can therefore be experimentally detected.

Additional amplification of electrical signal can be achieved by intercalating optically active complexes (intercalators) into the DNA. Examples of such intercalators include but are not limited to redox-active complexes, for example, Ru-complexes such as Ru(bpy)²⁺, Ru(bpy)³⁺, etc.

In a second embodiment of the invention, direct electrical measurements are made of electrical current flowing through a DNA-anchored nanoshuttle. A theoretical model of the shuttle mechanism for charge transfer in nanostructures is disclosed in a paper by L. Y. Gorelik et al, published in Phys. Rev. Lett. 80, 4526-4529 (1998). Such a nanoshuttle is shown in FIG. 2. Here two single strands of probe DNA 20 are interconnected through the nanoparticle 24. Their outer ends are connected to electrodes 22. Current is detected by resonance detector 25 capable of detecting the resonant frequencies of the nanoshuttle from the current flowing through it.

The charge-transfer behavior of the nanoshuttle is due to the phenomenon of Coulomb blockage, which refers to a suppression of current tunneling through metallic grains embedded in a dielectric matrix. The origin of this phenomenon lies in the quantization of charge in units of e (charge of one electron). For example, with reference to FIG. 2, if a size of the metallic nanoparticle (shadowed in FIG. 2) placed in between two electrodes is small enough, e.g. in the range of a few nanometers, an electrostatic charging energy of the nanoparticle (e²/2C, with the particle electrical capacitance C˜r, where r is the particle radius) can be large compared to other relevant energies related to temperature and bias voltage. The tunneling current through the particle is then blocked until the bias voltage V is increased to match the energy of tunneling electron with the energy level of the nanoparticle-trapped electron (V=e/C).

Gorelik et al. suggested that, if a dielectric material surrounding the nanoparticle is elastic and consists of mechanically soft organic molecules, a self-excitation of the nanoparticle mechanical vibrations, accompanied by a deformation of surrounding molecules, is possible. In that regime, the nanoparticle oscillates between two turning points. One of them is located near a positively, and the other near a negatively biased electrode (FIG. 2). Because of the Coulomb blockage phenomenon, an integer number of electrons are loaded onto the nanoparticle close to one turning point, and the same number of electrons are unloaded close to the other turning point. The result is that in each oscillation cycle the nanoshuttle moves a discrete number of electrons from one electrode to the other. Theoretically, different oscillation regimes were studied by Fedorets et al., Phys. Rev. Lett. 92, 166801 (2004). Experimentally, a nanoshuttle-like behavior was observed for a nanomechanical resonator with a “quantum bell” geometry by Erbe et al., Phys. Rev. Lett. 87, 096106 (2001).

In this embodiment of the present invention, single-stranded probe DNA molecules are used to anchor a metallic, semiconductor, or Fullerene nanoparticle between the electrodes (see FIG. 2). Nanoparticles of different size are now commercially available (e.g., 1.4 nm in diameter gold particles by Nanoprobes, Stony Brook, N.Y.). The nanoparticles can be attached to DNA molecules using a technique disclosed by, e.g., Alivisatos et al. (Nature, 382, 609-611 (1996)). A technique of attaching a DNA molecule to a gold electrode through sulphur-gold interaction was disclosed in a paper by Braun et al. (Nature, 391, 775-778 (1998)). A review of gold nanoparticle-labeled DNA molecules immobilized on a substrate was disclosed by Park et al. (Science 295, 1503-1506 (2002)).

Particular parameters, i.e., frequency, amplitude, number of shuttled electrons, etc., of the nanoshuttle oscillations depend on elastic constants of the DNA anchors. A hybridization of the single-stranded probe and the complementary target DNA molecules results in a change of the DNA anchor elastic constant, length, possibly conformation, etc. and, hence leads to a change of the nanoshuttle behavior, namely, the transmitted current amplitude and the nanoshuttle oscillation frequency. In a case of partial target-probe hybridization, the number of target-probe base pair mismatches affects the strength of coupling between the DNA strands and, hence, the DNA elastic properties. Thus, the number of mismatches can be quantified by measurements of the nanoshuttle current amplitude and spectral characteristics.

Instability and an oscillatory behavior of the nanoshuttle system as described above takes place when the elastic and electrical subsystems of the nanoshuttle exchange energy and support each other oscillations. The DNA molecules possess a pool of eigen-oscillation modes (which is due to a huge number of mechanical degrees of freedom including optical and acoustic types of vibrations, rotations, etc.) spanning from kilohertz to gigahertz frequencies. Thus, it is the electrical subsystem that picks the resonant elastic mode out of the pool, resulting in a characteristic resonance frequency of approximately 1/RC, where R is about the DNA Ohmic resistance and C is the capacitance of the nanoparticle. The capacitance of a nanometer size particle is in a range of 10⁻¹⁸-10⁻¹⁹ F. A typical DNA resistance is from 10¹⁰ to 10¹² Ohm depending on the length of the helix. Thus, the resonance nanoshuttle frequency is in a megahertz range and can be precisely experimentally determined.

The nanoshuttle oscillations can be modulated or optically excited. Photo-excitation of electromechanical oscillations can be easily demonstrated with a nanoshuttle made using a semiconductor nanoparticle or a Fullerene molecule as described above.

### EXAMPLE 2

To contact the molecules and perform nanoshuttle measurements a mechanically controllable break-junction technique according to van Ruitenbeek et al., Rev. Sci. Instrum. 67, 108 (1996) was used. A gold nanobridge was lithographically patterned on a phosphorus bronze substrate. Bending the substrate with a vertically moving rod causes the gold bridge to become elongated and finally break. The two resulting ends serve as electrodes for contacting the molecules.

Prior to the DNA adsorption the gold substrates were first rinsed twice in boiling ethanol for 20 min. After drying in nitrogen they were immersed in hot piranha solution (3:1 H_{—}2SO_{—}4:H_{—}2O) for 10 min. They were then thoroughly rinsed with ultra-pure water (Millipore, 18 MOhm). The 3′-thiolated DNA oligonucleotides were kept in their oxidized form-(CH_{—}2)_{—}3—S—S—(CH_{—}2)_{—}3—OH in order to protect the thiol group from undesired oxidation products or dimerization. Prior to adsorption, DNA was incubated with 10 mM of the reducing agent Tris(2-carboxyethyl) phosphine (TCEP) in 100 mM Tris-HCl, pH 7.5. The mixture was incubated at room temperature for several hours to allow complete reduction of the disulfide bond. The DNA samples were then passed through a column (BioSpin 6, BioRad) pre-equilibrated with the buffer (0.4 M NaH_{—}2PO_{—}4, pH 7.4). The final ssDNA concentration was adjusted to 10 μM. From 10 to 15 μl of the 10 μM reduced DNA solution was pipetted onto the clean gold surface.

The gold samples were then placed in a sealed Petri dish at 100% humidity. After 2 h of adsorption the samples were rinsed by 20 min incubation in the adsorption buffer. The rinsing was performed three times. Then, the samples were thoroughly rinsed with sterile ultra-pure water. Samples were kept in sterile ultra-pure water and were dried under nitrogen just prior to characterization. The DNA sequence used to form monolayer on the flat gold surface was: 5′-TAT-GCA-GAA-AAT-CTT-AG-3′. Gold nanoparticles (Aldrich-nominal, 10±3 nm diameter) were rinsed with de-ionized water by two centrifugations at 8000 rcf. After the water surfactant was removed, 200 μl of 10 μM of reduced thiolated ssDNA diluted in sterile deionized water was added to the gold nanoparticles. The mixture was stirred overnight at room temperature. Following incubation, the nanoparticles were rinsed with water and Tris (0.025 M)-NaCl (0.2 M pH 7.5) buffer by one centrifugation cycle at 10000 rcf. The nanoparticle-ssDNA complex in 0.2 M buffer was agitated for 4 h at room temperature, followed by rinsing twice in Tris (0.025 M)-NaCl (0.4 M).

The 5′CTA-AGA-TTT-TCT-GCA-TAG-CAT-TAA-TG-3′ sequence was used to form a monolayer on gold nanoparticles. 10 to 15 μl of the nanoparticle-ssDNA diluted in a Tris ç were dropped onto the ssDNA monolayer on gold break-junction electrodes. The sample was placed in a sealed Petri dish at 100% humidity bas above. After 12 h of incubation each sample was rinsed three times for 20 min, with the Tris (0.025 M)-NaCl (0.4 M) buffer. Prior to characterization, each sample was rinsed with sterile deionized water to remove excess salt. Electrical measurements were made using a Keithley Instruments Model 236 Source-Measure Unit; the output signal frequency was analyzed with an Agilent Technologies 53132A Universal Counter. Oscillations with the eigenfrequency around 110 MHz were detected when applied voltage exceeded a 4 V threshold. Entering into the oscillatory regime was also accompanied by the abrupt, manifold increase in the output current, which approached the 70 nA level. After addition of 10-15 μl solution containing the complementary ssDNA molecules (5′-CAT-TAA-TG-3′, 10 fM concentration) and a 10 min incubation, we observed a 3% increase in the oscillation frequency accompanied by the proportional increase in the current amplitude.

In this embodiment it is also possible to achieve improved results by metallizing the DNA, i.e., converting the nanoshuttle anchor DNA into the M-DNA form upon successful target-probe hybridization. The M-DNA formation is accompanied by strengthening the coupling between base pairs and, hence, by stiffening the double helix structure of the DNA. Thus, the change in DNA elastic coefficient upon the single-stranded DNA-M-DNA conversion and the resulting change in nanoshuttle oscillations are even more pronounced than that upon the conversion of ssDNA into a regular double-stranded B-DNA form. Besides, a direct current (DC) component of the nanoshuttle current can increase because of the DNA anchor metallization. As a result, this method provides high detection sensitivity of a hybridization event in both the DC and AC electrical measurements.

Examples will now be given of methods of forming single strand DNA. Optimized for ssDNA from phagemid pBluescript propagated in E.coli host XLI (Stratagene).

1. Triton Method: For manual sequencing with T7 polymerase and 35S-labelled ddATP, or cycle sequencing with Thermosequenase (Amersham), in combination with electrophoresis on the automatic sequencer Licor 4000L.

2. SDS Method: For cycle sequencing with Sequiterm (Epicentre) (or Thermosequenase (Amersham)), in combination with electrophoresis on the automatic sequencer Licor 4000L.

Triton Method

Media and Reagents

LB stock: 5×, filter sterilized, frozen.
Kanamycin stock: 10 mg/ml
Ampicillin stock: 100 mg/ml
Chloramphicol stock: 25 mg/ml
Tetracycline stock: 5 mg/ml
Culture medium: LB ½×
M9 salts 1×
Glycerol 1%
Tetracycline 5 μg/ml
Ampicillin 100 μg/ml.
PEG-solution: PEG Carbowax-8000 (20%);
Ammonium acetate (3.5 M), pH 7.5.
Triton stock: 10% (store at −20° C.)
Proteinase K stock: 10 mg/ml (store at −20° C.)
Triton/Prot.K-sol'n: Triton 0.1%
Tris 100 mM, pH 7-8
EDTA 5 mM
Proteinase K 100 μg/ml.
Mix freshly together before usage.
Helper phage K07 stock: 10exp12 pfu/ml stock
Culture containers: Cell Well culture plates (Corning 25820).

### Cultures

The day before inoculation of liquid cultures, transfer clones onto a fresh agar plate and incubate for 24 hrs at 37° C. For some libraries, if not older than 1 year, clones can be inoculated directly from the frozen microtiter plates.
1. Inoculate 100 μlmedium, by taking an amount of cells that corresponds to a microcolony (size =).
2. Shake cultures at 37° C. for 1 hr (=log culture of O.D.600=0.6.)
3. Add 2 μlhelper phage (=2×10exp9) and shake for 1 hr (up to 2) at 37° C.
4. Add 1.0 ml of LB supplemented with 70 μg/ml kanamycin and shake at 150 rpm for 16-18 hrs (up to 20) at 37° C.

II. Preparation of ssDNA
1. Pour culture in eppy tube and spin down for 10 min at 13000 rpm.
2. Transfer supernatant into new tube, add 300 μlPEG-sol'n.
3. Let sit for 10-15 min at rt.
4. Spin for 5 min at 13,000 rpm (gives smear or pellet), discard supernatant with suction.
5. Spin again for 1 min and remove carefully the rest of liquid.
6. Add 50 μl Triton/Prot.K-sol'n, let sit for 5-15 min (rt or 37° C.), vortex briefly, incubate at 37° C. for 30 min, vortex again. Load an aliquot of 2 μlon an agarose gel to check size and yield (loading buffer must contain 0.2% SDS for better EB staining). Continue incubation for another 30 min.
7. Heat-denature Prot.K for 10 min at 90° C., then chill on ice and spin down for 2-5 min. Take 6 μl of the supernatant for a full sequencing reaction.
(It is not necessary to transfer the supernatant to new tube).
   III. Storage of ssDNA

For sequencing purposes, the DNA quality is stable for at least 1 year of storage at +4° C. or −20° C.
When stored at 4° C., compensate for evaporation by adding H2O to restore the original volume. Before using the DNA for a sequencing reaction, mix the DNA solution, spin turbidity down briefly and take an aliquot from the supernatant. When stored at −20° C., repeat step II.7 before using the DNA for a sequence reaction.
   SDS Method

Media and Reagents
LB stock: 5× LB, filter-sterilized (stored at −20° C.)
Ampicillin stock: 100 mg/ml (in H2O)
Tetracycline stock: 5 mg/ml (in 50% ethanol)
Kanamycin stock: 70 μg/ml (in H2O)
Culture medium: ½×LB, 1×M9 salts, 1% glycerol,
100 μg/ml ampicillin, 5 μ/ml tetracycline.
Culture “tubes”: Multiwell tissue culture plates Falcon 3047.
PEG-sol'n: PEG Carbowax 8000 (20%), 3.5 M ammonium acetate, pH 7.5.
SDS/Prot.K-sol'n: 0.1% SDS, 100 mM Tris, 5 mM EDTA, pH 7-8; 100 μg/ml Proteinase K (add Prot.K only before usage.)
   Culture

24 hrs before inoculation, transfer clones onto a fresh agar plate, containting ampicillin and tetracycline.
1: Inoculate clones into 100 μl medium, by taking a tiny amount of cells that corresponds to a micro colony (size of this “.” and not more!).
2. Shake cultures at 37° C. for 1 hr to obtain a log culture of ˜O.D.600=0.6. Add 2×10exp9 helper phage.K07 (2 μgl of 10exp12 pfu/ml stock) and shake at 37° C. for 1 hr at 150 rpm.
3. Add 1.0 ml LB supplemented with 70% μg/ml kanamycin and shake at 37° C. for another 16-18 hrs. Less turbid cultures should be shaken some extra hours.
   II. Preparation of ssDNA
1. Pour culture in eppy tube and spin down for 10 min 13000 rpm
2. Pour supernatant in new tube and add 300 μl PEG-sol'n.
3. Let sit 10 min at room temperature.
4. Spin down for 5 min at 13,000 rpm (gives smear or pellet), discard supernatant with suction.
5. Spin down again for 1 min and remove carefully all residual liquid.
6. Dissolve pellet thoroughly by vortexing in 300 μlSDS/Prot.K-sol'n, incubate at 37° C. for 1 hr, or at room temp. o.n.
7. Check size of ssDNA on agarose gel (load 5 ul).
   III. Purification of ssDNA
1. NaCl extraction: add 75 μlNaCl of a 5 M stock solution to ssDNA (gives 1 M final), put for 1 hr on ice, spin for 10 min, turn tube for 180° and spin again for 10 min. Transfer supernatant immediately into new tubes.
2. Precipitate ssDNA with ethanol+3M ammonium acetate, put on ice for 10 min, spin for 5 min at 13,000 rpm at 4° C.
3. Drip-dry pellet, wash it with 70% ethanol, drip-dry, then dry for several min at 60° C.
4. Re-dissolve pellet in 12-20 μlTE by vortexing (DNA sticks all over the tube). Yield: up to 10 μg of DNA. Store at −20° C.

All references are herein incorporated by reference.

We claim:

1. A method of detecting the presence of a match of a target DNA base sequence with a probe DNA base sequence, comprising:
preparing a single strand of the probe DNA base sequence;
linking a first end of said single strand probe DNA base sequence to an electrode and a second end to a nano entity capable of exchanging charge with the DNA base sequence;
bringing a single strand of the target DNA base sequence into contact with the single strand of the probe DNA base sequence; and
detecting a change in a physical property of said probe DNA base sequence due to hybridization in the event of a match of and said target DNA base sequence.

2. A method as claimed in claim 1, wherein the transfer of charge between the DNA base sequence and the nano entity is detected upon hybridization.

3. A method as claimed in claim 1 or 2, wherein said nano entity is a fullerene molecule, a nanoparticle or a redox-active molecule.

4. A method as claimed in any one of claims 1 to 3, wherein said entity is optically excited to initiate charge transfer.

5. A method as claimed in any one of claims 1 to 4, further comprising applying an electrical voltage to the electrode and detecting the current generated upon hybridization.

6. A method as claimed in any one of claims 1 to 5, wherein said single strand probe DNA sequence is bonded to said electrode with the aid of a monolayer of said single strand probe sequence deposited on said electrode.

7. A method as claimed in claim 6, wherein said electrode is a gold electrode on an semiconductor substrate.

8. A method as claimed in any one of claims 1 to 7, wherein said entity is linked to the second end of the single strand of probe DNA base sequence with a linker molecule.

9. A method as claimed in any one of claims 1 to 8, further comprising metallizing said single strand DNA probe sequence to enhance conductivity upon hybridization.

10. A method as claimed in claim any one of claims 1 to 8, further comprising adding an intercalator consisting essentially of a solution of divalent metal ions having a d-electron shell to intercalate any double strand DNA formed by hybridization.

11. A method as claimed in claim 10, wherein the single strand probe DNA base sequence is brought into contact with the single strand target DNA base sequence in the presence of said intercalator so that the hybridized DNA is intercalated by said divalent metal ions.

12. A method as claimed in any one of claims 1 to 3, wherein a pair of said single strands of the probe DNA base sequence are interconnected at their respective second ends by said entity and are anchored at their first ends to respective electrodes to create a nanoshuttle.

13. A method as claimed in claim 12, wherein said physical property is an oscillatory property of the nanoshuttle.

14. A method as claimed in claim 13, wherein the resonant frequency of the nanohuttle is detected.

15. A method as claimed in any one of claims 12 to 14, wherein the entity is optically excited to enhance said change.

16. A method as claimed in claim any one of claims 12 to 15, further comprising adding an intercalator consisting essentially of a solution of divalent metal ions having a d-electron shell to intercalate any double strand DNA formed by hybridization.

17. A method as claimed in claim 16, wherein the single strand probe DNA base sequence is brought into contact with the single strand target DNA base sequence in the presence of said intercalator so that the hybridized DNA is intercalated by said divalent metal ions.

18. A DNA detection apparatus, comprising:
a sensor with a surface forming an electrode;
a single strand probe DNA base sequence having one end bonded to an electrode and another end linked to an entity selected from the group consisting of a fullerene molecule, a nanoparticle, and a redox-active molecule; and
a detector for detecting current flowing through the DNA base sequence upon hybridization.

19. An apparatus as claimed in claim 18, further comprising a source for optically exciting said entity.

20. An apparatus as claimed in claim 18 or 19, wherein the single strand proble DNA base sequence is attached to said electrode by a monolayer of said DNA probe base sequence.

21. An apparatus as claimed in claim 18, comprising a pair of single strands of said DNA probe base sequence having first ends interconnected through said entity and second ends attached to respective electrodes to form a nanoshuttle.

22. An apparatus as claimed in claim 21, further comprising a detector for detector resonance frequencies of said nanoshuttle.

22. An apparatus as claimed in any one of claims 18 to 21, further comprising an intercalator for said DNA base sequence.

23. An apparatus as claimed in claim 22, wherein said intercalator comprises a solution of divalent metal ions having d-electron shell.

24. An apparatus as claimed in any one of claims 21 to 23, further comprising a source for optically exciting said entity.

25. A method of detecting the presence of a match of a target DNA base sequence with a probe DNA base sequence, comprising:
preparing at least first and second single strands of the probe DNA base sequence;
linking one end of each of said first and second single strands of probe DNA base through a nano entity capable of exchanging charge with the DNA base sequence to create a nanoshuttle;
bringing a single strand of the target DNA base sequence into contact with at least one of the single strands of the probe DNA base sequence; and
detecting a change in a physical property of said probe DNA base sequence due to hybridization in the event of a match of and said target DNA base sequence.

26. A method as claimed in claim 25, wherein the detected change is the change in resonant frequency.
